(19) United States
(12) Patent Application Publication
(10) Pub. No.: US 2004/0077720 A1
(43) Pub. Date: Apr. 22, 2004

(54) Inhibition of antibody production employing perfluorooctanoic acid or derivatives thereof

(76) Inventors: Joseph William Depierre, Stockholm (SE); Sven jan Lennart Nassberger, Lund (SE)

Correspondence Address: SMITH, GAMBRELL & RUSSELL, LLP, 1850 M STREET, N.W., SUITE 800, WASHINGTON, DC, 20036, US

(21) Appl. No.: 10/406,011

(22) Filed: Apr. 3, 2003

(30) Foreign Application Priority Data
Apr. 5, 2002 (SE) 0102441-3

(51) Int. Cl. A61K 31/20

(52) U.S. Cl. 514/558

(57) ABSTRACT

The invention relates to a substance, and its derivatives, which specifically inhibits production of autoantibodies. Both immune responses to active immunization and endogenous production of autoantibodies are inhibited. Consequently, no circulating immunocomplexes which can give rise to deposits in tissues/organs are formed. In parallel, there is total blockage of the development of lymphoadenopathy in the form of lymphocyte proliferation.

This application claims priority to Swedish Priority Document Application No. 0102441-3, filed on Apr. 5, 2002. The Swedish Priority document is hereby incorporated by reference in its entirety.

### BACKGROUND OF THE INVENTION

In the presence of high levels of circulating antibodies, immunocomplexes may be formed and deposited in various organs. The latter occurs when the capacity of the reticuloendothelial system to clear immunocomplexes is exceeded. The organs most commonly affected include the kidneys, lungs and intestines. Such depositions in the kidneys can result in so-called immunocomplex-mediated glomerulonephritis. The deposits are seen in the glomerulus and blood vessels and give rise to vasculitis, i.e., inflammation in the walls of the blood vessels.

Furthermore, a variety of autoimmune diseases are associated with polyclonal stimulation of B-lymphocytes, resulting in high levels of circulating antibodies. One such example is the disease systemic lupus erythematosis. Thus, overstimulation of the immune system with rapid proliferation of different types of lymphocytes (e.g., B-lymphocytes, autocytotoxic T-lymphocytes) plays an important role in the development of autoimmune disease. The development of mature lymphocytes with specific functions from immature cells takes place continuously. Substances which inhibit this developmental process are of potentially great value for treatment of autoimmune diseases.

Other specific diseases in which circulating autoantibodies are considered to be causal agents include diabetes mellitus type I, in which circulating antibodies are directed against the enzyme glutamic acid dehydrocarboxylase. Another example is myeloma, a malignant disease characterized by proliferation of plasma cells and B-lymphocytes in different stages of development. In this case an M component, i.e., an extremely high level of immunoglobulins, in particular IgG or IgM, is observed.

### BRIEF SUMMARY OF THE INVENTION

Embodiments of the present invention provide pharmaceutical compositions and methods of treatment.

One embodiment of the present invention concerns a pharmaceutical composition for inhibiting antibody production in a reversible fashion comprising an antibody inhibiting effective amount of perfluorooctanoic acid or derivative thereof and a pharmaceutically acceptable carrier.

Another embodiment concerns a pharmaceutical composition, comprising an effective amount of perfluorooctanoic acid or derivative thereof and a pharmaceutically acceptable carrier, wherein said effective amount is an amount effective to inhibit the formation of immunocomplexes in a reversible fashion, inhibit development of immature lymphocytes into mature lymphocytes in a reversible fashion, inhibit lymphocyte proliferation in a reversible fashion, prevent growth of normal lymph nodes into pathologically enlarged lymph nodes, or inhibit production of IgG or IgM associated with myeloma and benign monoclonal gammapathy.

Yet another embodiment concerns a method of inhibiting antibody production in a reversible fashion comprising administering to a subject in need thereof a composition including an antibody inhibiting effective amount of perfluorooctanoic acid or derivative thereof and a pharmaceutically acceptable carrier.

An additional embodiment concerns a method of treating a disease comprising administering to a subject in need thereof a disease treating effective amount of perfluorooctanoic acid or derivative thereof and a pharmaceutically acceptable carrier.

### BRIEF DESCRIPTION OF THE DRAWINGS

FIG. 1 shows a chart showing the development of stem cells and at what step in development PFOA is thought to have an effect;

FIG. 2 is a picture of a mouse from the control group of the Example;

FIG. 3 is a picture of a mouse treated with compound A from the Example;

FIG. 4 is a picture of a mouse treated with compound B from the Example;

FIG. 5 is a graph showing protein concentrations in urine samples from the mice from the Example;

FIG. 6 is a graph showing protein concentrations in urine samples from the mice from the Example;

FIG. 7 is a table showing the effects of compounds A and B on body weight and the weight of various organs from the Example;

FIG. 8 shows a comparison of lymph nodes taken from mice from the three test groups from the Example;

FIG. 9 shows a comparison of thymus taken from mice from the three test groups from the Example;

FIG. 10 shows a comparison of spleen taken from mice from the three test groups from the Example;

FIG. 11 shows a comparison of kidney taken from mice from the three test groups from the Example;

FIG. 12 shows a comparison of liver taken from mice from the three test groups from the Example; and

FIG. 13 is a graph showing protein concentrations in urine samples from mice treated with compound B from the Example after treatment was stopped.

### DETAILED DESCRIPTION OF THE INVENTION

The present invention concerns a substance, and its derivatives, which specifically inhibits production of autoantibodies. The invention also concerns a fluorine containing aliphatic carboxilic acid compound having the formula:

CX3(CX2)ₙCOOH. (I)

In the above formula at least one X is a fluorine and the remaining Xs are at least one of fluorine, chlorine, bromine, iodine, hydrogen, or a methyl group. Moreover, n can be from 3 to 6. Perfluorooctanoic acid is preferred.

Applicants discovered that both immune responses to active immunization and endogenous production of autoantibodies are inhibited by the compound of the present invention. Consequently, no circulating immunocomplexes which can give rise to deposits in tissues/organs are formed. In parallel, there is total blockage of the development of lymphoadenopathy in the form of lymphocyte proliferation. These effects are mediated either via nuclear receptors and/or induction of FAS-mediated apoptosis.

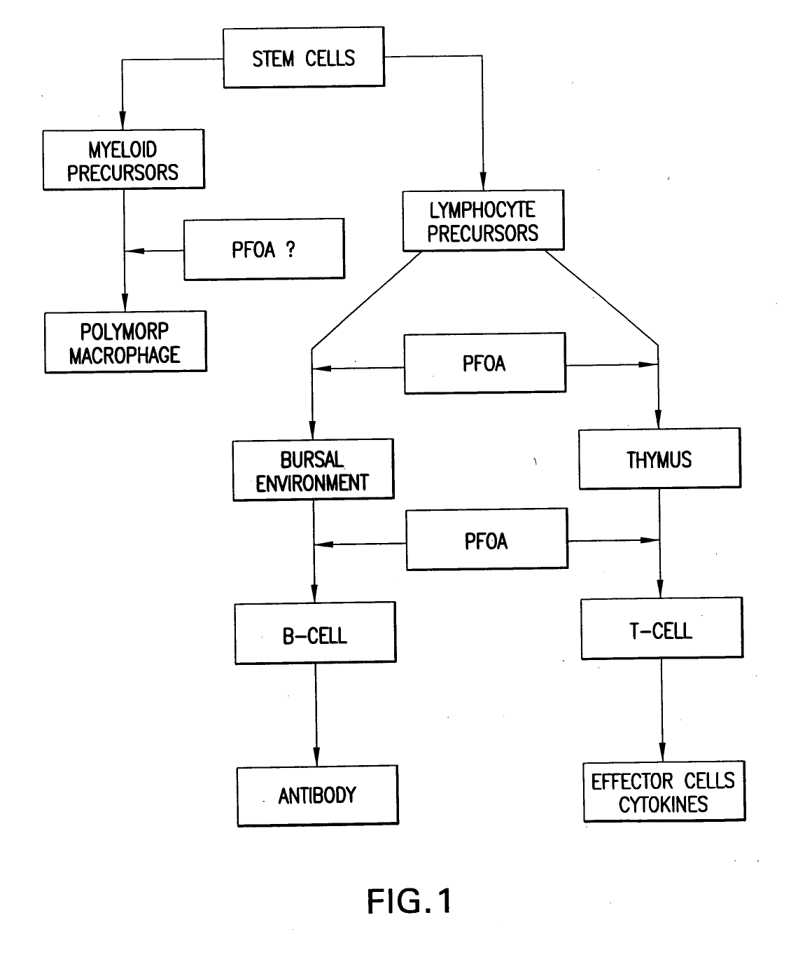

Perfluorooctanoic acid (PFOA) and its derivatives, as represented by formula (I), inhibit production and development of mature immune cells from stem cells or myeloid or lymphocyte precursors stage respectively. With reference to FIG. 1, most likely PFOA inhibits the step after the precursors stage. This inhibition is reversible, or in other words, when PFOA is withdrawn the differentiation process continues, or returns to normal.

PFOA belongs to a group of chemicals that induce peroxisome proliferation in the liver in rodent. During laboratory studies, a reduction of thymus and spleen was observed. These initial observations led to the inventors' discovery that PFOA could interfere with the immune system, and that PFOA could express immunosuppressive properties. Furthermore, it has been demonstrated that neonatal thymectomy has a significant suppressive effect on this disorder in MRL mice.

Without wishing to be bound be any particular theory, the mechanism of PFOA involves inhibition of lymphocyte proliferation and direct inhibition of antibody production, as has been shown in experimental studies Applicants have performed. This has been demonstrated using a special strain of mice designated MRL 1 pr/1 pr (MRL/ML-Tnfrs6/1 pr). The lupus-like disease in these MRL-mice is characterized by a number of iuumunological abnormalities, including hypergammaglobulinemia, production of a variety of autoantibodies, circulating immunocomplexes and reduced serum levels of complement. Accordingly, this mouse strain is used as a model for human systemic lupus erythematosis.

In the MRL-strain, half of the mice die at age 5 months. Three to four months later, 100% mortality is seen. If a compound displays an effect it will be rapidly observed by an increased survival rate and clinical well-being. Applicants have found that treated mice have a significant increased survival rate compared to untreated ones.

The MRL-mouse is a genetic model of systemic lupus erythemathosus (SIF), a prototypical autoimmune disease in humans. The MRL-mouse strain is the best animal approximation model to study this human disease, and therefore in common use world-wide. The strain has been used to investigate several immunosuppressive drugs, such as cyclosporin, FK 506 and rapamycin, which are in clinical use today.

Articles that include MRL-strain as an experimental model include Warner L. M., Adams L. M., Sehgal S. N. Rapamycin prolongs survival and arrests pathological changes in murine systemic lupus erythematosus, Arthritis & Rheumatism, 17, 289-297, 1994; Yamamoto K., Mori A., Nakahama T., Ito M., Okudaira H., Miyamoto T. Experimental treatement of autoimmune MRL-1 pr/1 pr mice with immunosuppressive compound FK 506, Immunology 69, 222-227, 1990; Bartlett R. R., Popovic S., Raiss R. X. Development of autoimmunity in MRL/1 pr mice and the effects of drugs on this murine disease, Scand. J. Rheumatology Suppl. 75, 290-299, 1988; Kiberd B. A., Young I. D. Modulation of glomerular structure and function in murine lupus nephritis by methylpredinsolone and cyclophosphamide, J. Lab. Clin. Med. 124(4), 496-506, 1994; Haug L. Theofilopoulos A. N., Dixon F. J., A spontaneous rheumatoid arthritis-like disease in MRL-mice, J. Exp. Med. 155, 1690-1701, 1982; and Smith H. R., Chused T. M., Steinberg A. D., Cyclosphosphamide-induced changes in the MRL-1 pr/1 pr mouse: effects upon cellular composition, immune function and disease. Clin. Immunology and Immunopathology 30; 51-61, 1984. The above articles are hereby incorporated by reference in their entirety.

As the experimental animal model for studies of inhibition of disease development and the underlying mechanism(s), Applicants have employed MRL-Tnfrs6/1 pr mice, which spontaneously develop high levels of a wide range of antibodies and an SLE-like syndrome, including severe lymphoadrenopathy. The MRL-mouse strain is known to develop a severe autoimmune disease syndrome. In fact, it is known to develop the most severe disease state. Highs levels of circulating autoantibodies, immunocomplexes mediated glomerulonephritis, arthritis and vasculitis are observed. The mice also develop a heavy proliferation of lymph nodes.

The presence of circulating immunocomplexes is analyzed indirectly by continuous monitoring of the levels of protein, hemoglobin and erythrocytes in the urine of these animals, the quantities of which are directly related to the severity of the immunocomplex-mediated glomerulonephritis which develops. The absence of these substances in urine indicates that no immunocomplexes have been deposited in the kidneys and, thus, that no circulating immunocomplexes have been formed. When treatment of these mice with perfluorooctanoic acid is terminated, the animals develop disease symptoms, including elevated levels of circulating antibodies, formation of immunocomplexes and growth of lymph nodes. The effectiveness of a low dose of the substance and 100% reproducibility argue for a direct inhibition of the mechanism(s) underlying development of the disease.

The symptoms which these mice develop include deterioration of joints and consequent motor difficulties, kidney damage with resulting uremia (retention of fluid) and extreme lymphoadenopathy. This lymphoadenopathy arises from defects in the FAS antigen gene resulting from the lymphoproliferation (1 pr) mutation in MRL-mice. In the final stages of their disease these animals also develop dermal vasculitis. 100% of these mice develop these symptoms.

The degree of renal damage can be monitored on the basis of the level of protein, erythrocytes, hemoglobin and hyalin cylinders in the urine. Lymphocyte proliferation can be assessed from the growth of lymph nodes in the shoulders and groin, as well as along the aorta. Blood and tissue samples are collected for analysis and histopathological examination.

When these mice are administered perfluorooctanoic acid, none of these disease symptoms are observed. By inhibiting antibody production, this substance also prevents the formation of immunocomplexes. These treated animals appear completely normal, without joint deterioration, lymphocyte proliferation, dermal vasculitis or immunocomplex-mediated glomerulonephritis. When this substance is withdrawn, the mice begin to develop typical disease symptoms. These results have been shown to be 100% reproducible in different studies. In other experimental studies in which mice have been treated with perfluorooctanoic acid and subsequently challenged by immunization with horse erythrocytes, no antibody response to these foreign antigens was observed. These results led us to investigate whether this compound and its derivatives cause similar responses in animals with inborn (genetic) disorders in their immune system.

Diseases which can be treated by the following invention include vasculitis (inflammation of vessels) arthritis and autoimmune diseases especially where specific autoantibodies play a role in the pathogenic mechanism(s) such as diabetes mellitus type I and immune complex mediated glomerulonephritis. Other disease which can be treated by the present invention include diseases where the underlying pathogenic mechanism may be due to antigen/antibody complex. Yet other diseases which can be treated include diseases involving blood malignancies and denominated gammapathies. These diseases are characterized by high production of identical immunoglobulins. This overproduction of immunoglobulins results in an M-component (M=monoclonal). Examples of such diseases include myeloma, Morbus Waldenström and benign monoclonal gammapathy.

Treatment will be in the range of 10-15 mg/kg body wt/24 hrs for acute treatment and 0.1-2 mg/kg body wt/24 hours for long term treatment. The dosages can be administered orally once or twice per 24 hours. In the experimental model, the carrier chow R3 was used. However, for human usage, any well known pharmaceutically acceptable carrier, such as and oil or cremophor, can be used. The carbon chain of the PFOA compound can be lengthened or shortened by 1, 2, or 3 carbon atoms and still express potency. Furthermore, one to seven fluorine atoms can be replaced by a hydrogen atom/methyl group, and/or another halogen, such as —Cl, —Br, or I, and still express potency.

### EXAMPLE

Animal Model: MRL/MpJ-Tnfrsf6^{1pr}-Mice

This strain develops spontaneous systemic lupus erythematosus-like autoimmune syndrome at or about 8 weeks of age. The syndrome is characterized by massive lymphadenopathy, immune complex glomerulonephritis, arthritis, high level of circulating antibodies, and vasculitis.

Compounds A and B were tested. Compound A was a reference compound and compound B was PFOA. Treatment was initiated at the age of 5 and 8 weeks, respectively. All animals received the respective compounds on a daily basis. A control group of mice without treatment was run in parallel. The following parameters were screened.

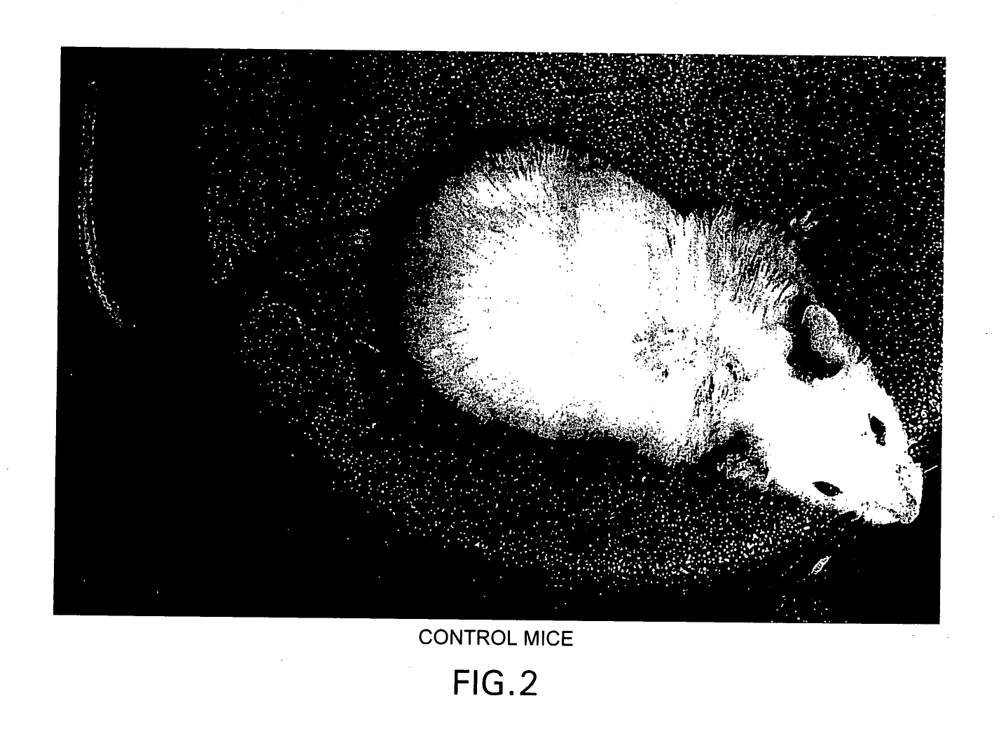

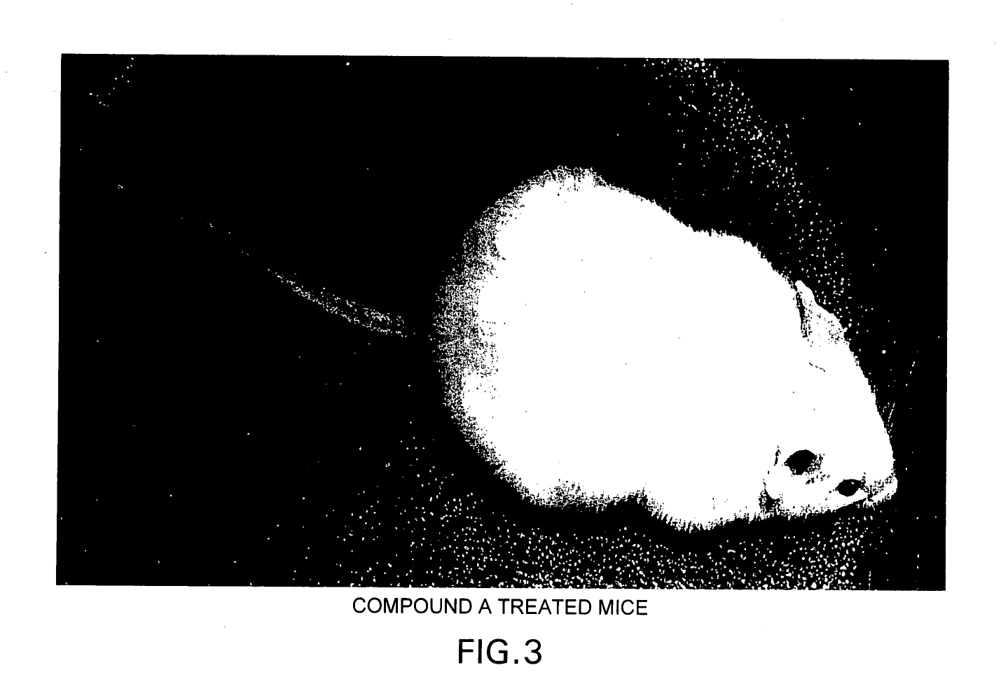

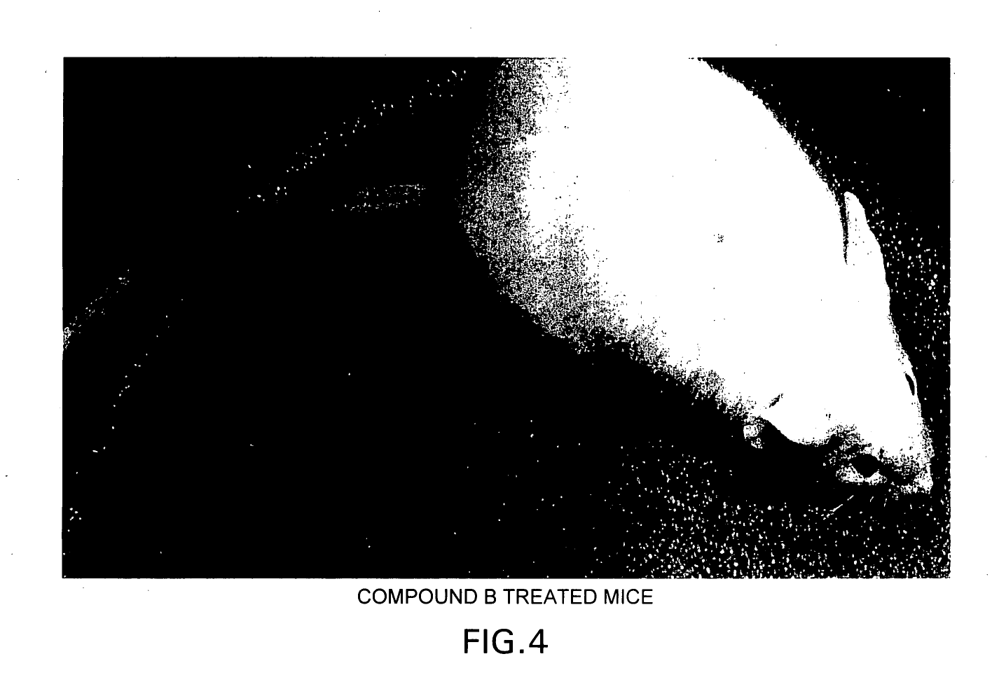

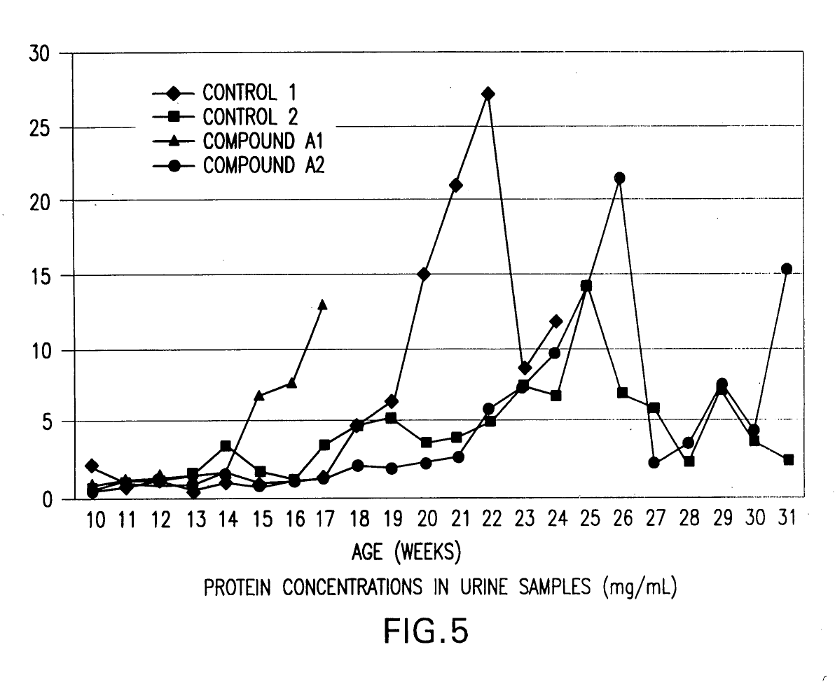

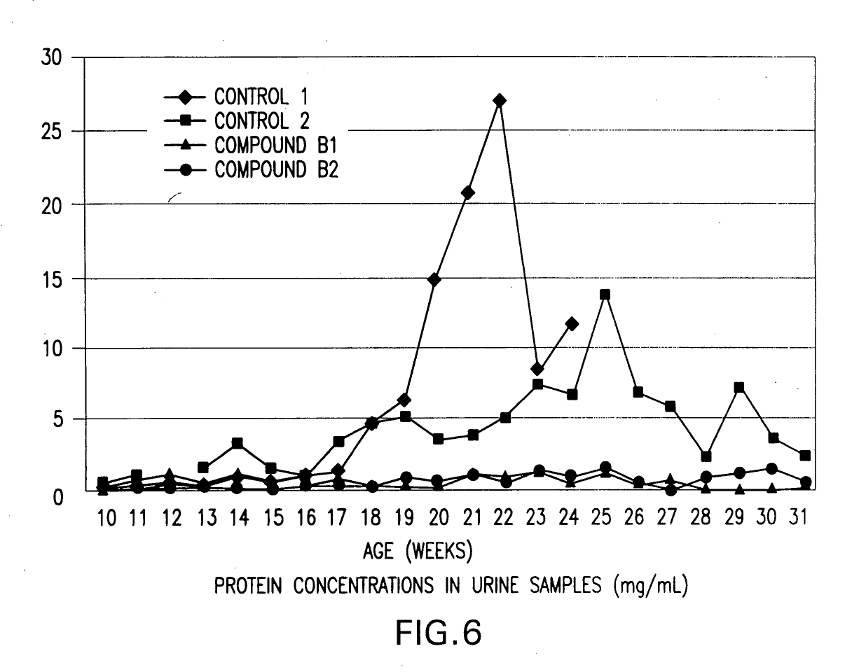

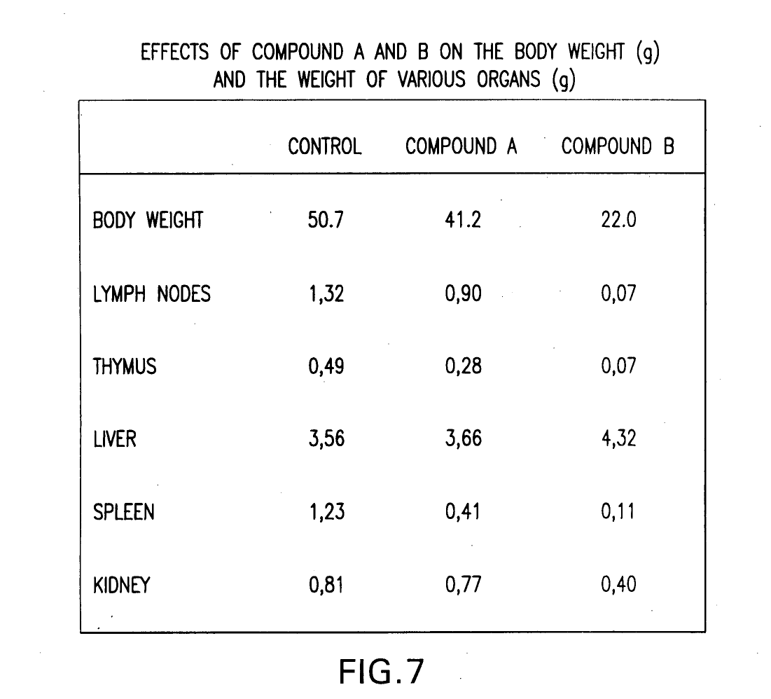

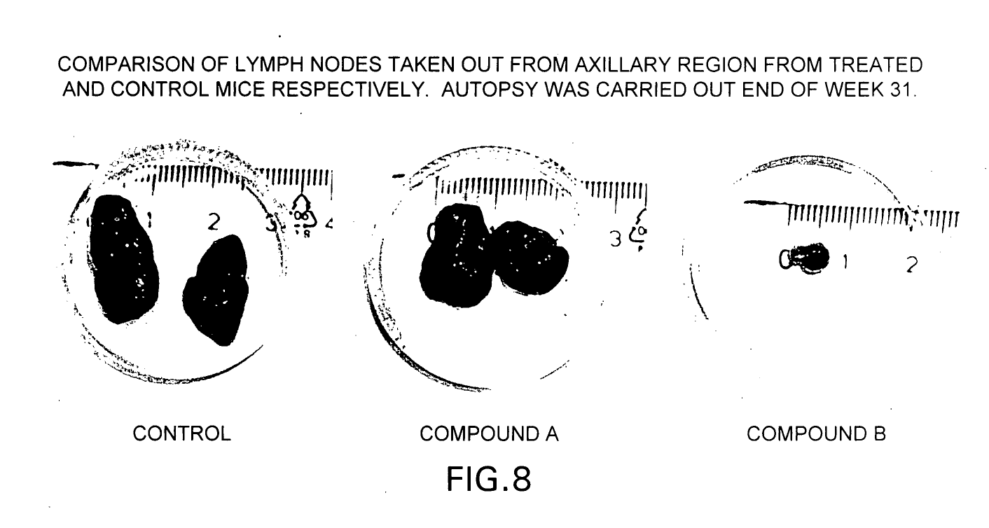

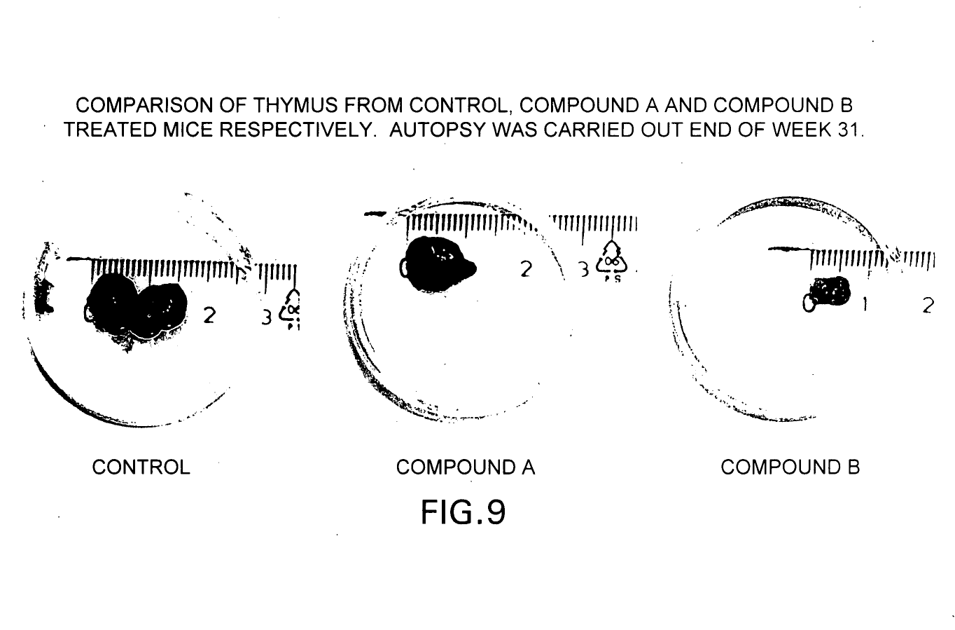

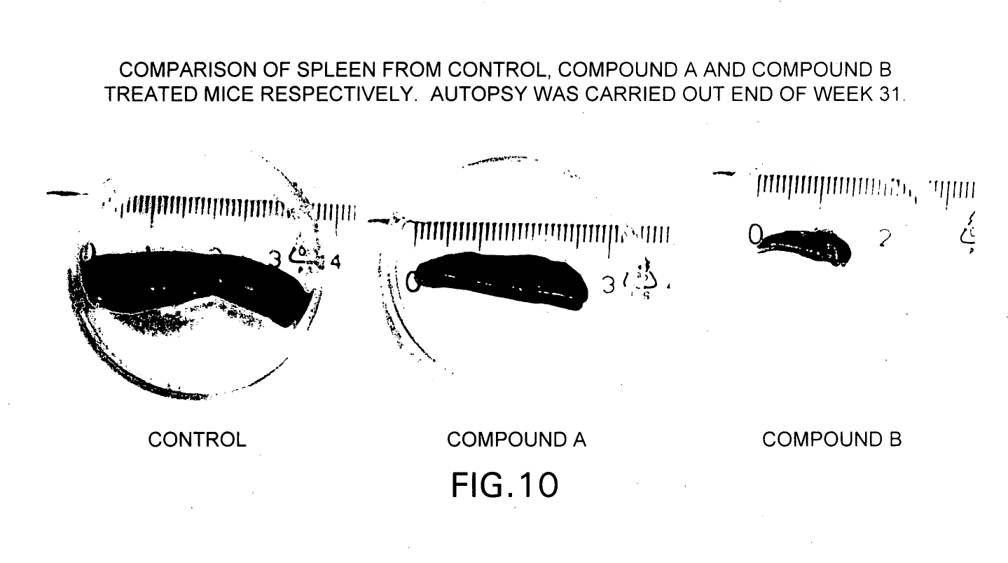

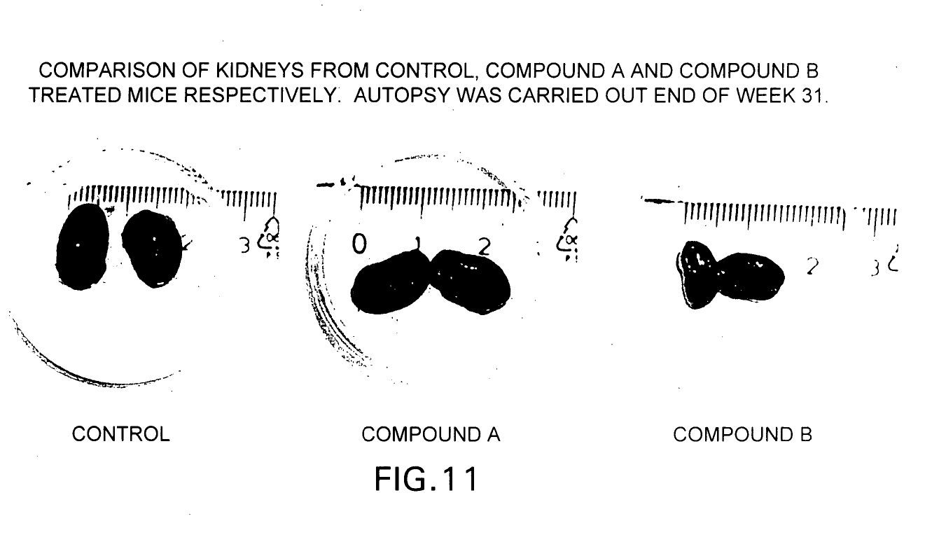

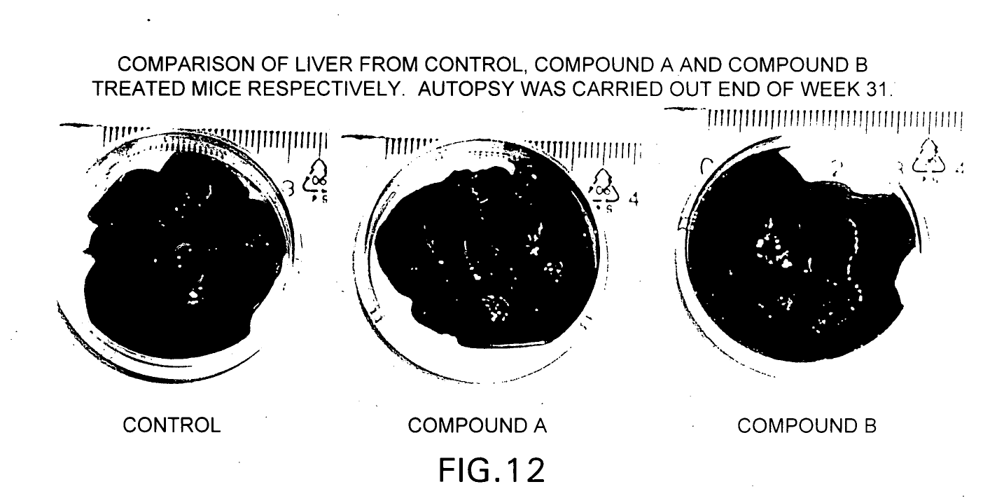

1. Clinical observation. Results shown in FIGS. 2-12.

2. Investigation of urine sediment.

3. Monitoring of proteinuria.

Experimental design

| | Age | Age |
|---|---|---|
| Compound A | Week 5 | Week 8 |
| Compound B | Week 5 | Week 8 |
| Control | | Week 8 |

Parameters followed during the study: 1) Clinical observation; 2) Urine: light microscopy, proteinuria. The following organs were sampled: thymus, lymph nodes, kidney, liver, and spleen. The organs were investigated by histopathology and immunohistopathology.

Results:

Control Mice:

From a clinical point of view, an increased body weight up to 50 g was observed. Furthermore, the mice had difficulty moving around and most of the time were resting. Bad fur was developed late in the disease. Severe dermal and ear ulcers were noted. At 16 weeks, enlarged lymph nodes were observed and continued to increase in size. At weeks 14-18, proteinuria was detected. At autopsy, anasarca was noted.

Compound A Treated Mice:

A body weight increase was found. At 16 weeks, visible lymph nodes were observed. During weeks 15-21, proteinuria was observed. From a clinical point of view, the mice moved slowly, but were more active when compared to controls. The fur was well-nourished as in normal mice. Small ulcers in the ears were seen occasionally. At autopsy, anasarca was detected.

Compound B Treated Mice:

From a clinical point of view, these mice were very active. The appeared with normal fur. No ulcers were observed. No body weight gain, proteinuria, or enlarged lymph nodes was recorded. At autopsy, no anasarca was detected.

Conclusions:

The control mice developed clinical signs of disease at age 14-16 weeks.

Mice treated with compound A developed signs of disease at around 20 weeks of age. However, it was found that compound A reduced the severity of this disease as observed from a clinical point of view, as well as the lymph nodes were smaller.

Mice treated with compound B did not develop any signs of disease. No lymphadenothapy was observed. A clear-cut increase of survival rate was demonstrated compare to the mice of the control group and mice treated by compound A.

Withdrawal Study:

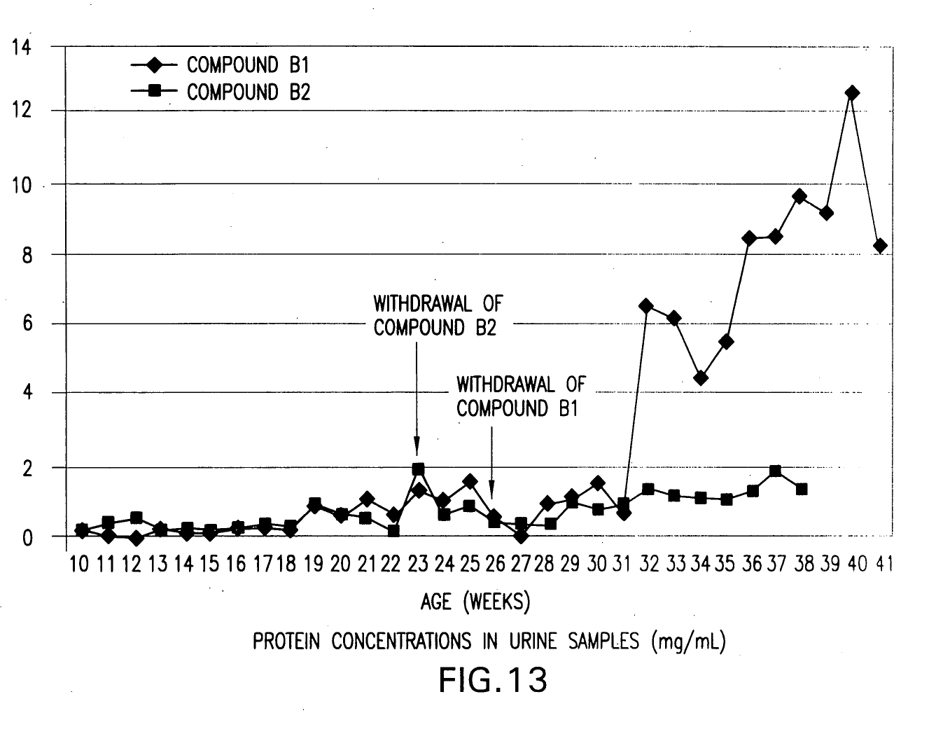

In a separate study compound B was withdrawn from mice treated during 23-26 weeks. The mice were followed for up to 39-42 weeks. From a clinical point of view no sign of symptoms were observed up to the 42 week point. However, an increase in weight was observed. Proteinuria was observed at 5 weeks after withdrawal of the substance. These results are shown in FIG. 13.

It should be emphasized that the above-described embodiments of the present invention, particularly, and “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of the disclosure and the present invention and protected by the following claims.

We claim:

1. A pharmaceutical composition for inhibiting antibody production in a reversible fashion comprising an antibody inhibiting effective amount of perfluorooctanoic acid or derivative thereof and a pharmaceutically acceptable carrier.

2. The pharmaceutical composition according to claim 1, wherein the composition is in an amount effective for inhibiting antibody production associated with diabetes mellitus type I.

3. The pharmaceutical composition according to claim 1, wherein the composition is in an amount effective for inhibiting antibody production associated with rejection due to xeno-transplantation

4. A pharmaceutical composition comprising an effective amount of compound having the following formula:
CX3(CX2)ₙCOOH
wherein said effective amount is an amount effective to inhibit antibody production in a reversible fashion, inhibit the formation of immunocomplexes in a reversible fashion, inhibit development of immature lymphocytes into mature lymphocytes in a reversible fashion, inhibit lymphocyte proliferation in a reversible fashion, prevent growth of normal lymph nodes into pathologically enlarged lymph nodes, or inhibit production of IgG or IgM associated with myeloma and benign monoclonal gammapathy.

5. The pharmaceutical composition according to claim 4, wherein at least one X is a fluorine and the remaining Xs are at least one of fluorine, chlorine, bromine, iodine, hydrogen, or a methyl group.

6. The pharmaceutical composition according to claim 4, wherein n is from 3 to 6.

7. A pharmaceutical composition, comprising an effective amount of perfluorooctanoic acid or derivative thereof and a pharmaceutically acceptable carrier,
wherein said effective amount is an amount effective to inhibit the formation of immunocomplexes in a reversible fashion, inhibit development of immature lymphocytes into mature lymphocytes in a reversible fashion, inhibit lymphocyte proliferation in a reversible fashion, prevent growth of normal lymph nodes into pathologically enlarged lymph nodes, or inhibit production of IgG or IgM associated with myeloma and benign monoclonal gammapathy.

8. A method of inhibiting antibody production in a reversible fashion comprising administering to a subject in need thereof a composition including an antibody inhibiting effective amount of perfluorooctanoic acid or derivative thereof and a pharmaceutically acceptable carrier.

9. The method according to claim 8, wherein the composition is in an amount effective for inhibiting antibody production associated with diabetes mellitus type I.

10. The method according to claim 8, wherein the composition is in an amount effective for inhibiting antibody production associated with rejection due to xeno-transplantation

11. The method according to claim 8, wherein a dosage in an amount of about 10-15 mg/kg body wt/24 hrs is administered to the subject in need thereof.

12. The method according to claim 8, wherein a dosage in an amount of about 0.1-2 mg/kg body wt/24 hrs is administered to the subject in need thereof.

13. The method according to claim 8, wherein the composition is administered orally once or twice per 24 hours.

14. A method of treating a disease comprising administering to a subject in need thereof a disease treating effective amount of perfluorooctanoic acid or derivative thereof and a pharmaceutically acceptable carrier.

15. The method according to claim 14, wherein said effective amount is an amount effective to inhibit the formation of immunocomplexes in a reversible fashion, inhibit development of immature lymphocytes into mature lymphocytes in a reversible fashion, inhibit lymphocyte proliferation in a reversible fashion, prevent growth of normal lymph nodes into pathologically enlarged lymph nodes, or inhibit production of IgG or IgM associated with myeloma and benign monoclonal gammapathy.

16. A method of treating a disease comprising administering to a subject in need thereof a disease treating effective amount of a compound having the following formula:
CX₃(CX₂)ₙCOOH, and
a pharmaceutically acceptable carrier,
wherein said effective amount is an amount effective to inhibit antibody production in a reversible fashion, inhibit the formation of immunocomplexes in a reversible fashion, inhibit development of immature lymphocytes into mature lymphocytes in a reversible fashion, inhibit lymphocyte proliferation in a reversible fashion, prevent growth of normal lymph nodes into pathologically enlarged lymph nodes, or inhibit production of IgG or IgM associated with myeloma and benign monoclonal gammapathy.

17. The method according to claim 16, wherein at least one X is a fluorine and the remaining Xs are at least one of fluorine, chlorine, bromine, iodine, hydrogen, or a methyl group.

18. The method according to claim 16, wherein n is from 3 to 6.
